(19) United States
(12) Patent Application Publication
(10) Pub. No.: US 2024/0082475 A1
(43) Pub. Date: Mar. 14, 2024

(54) EXTRACORPOREAL PLASMA SEPARATION, ADSORPTION, AND DELIVERY SYSTEMS AND METHODS

(71) Applicant: CytoStorm, Inc., Laguna Hills, CA (US)

(72) Inventor: Lon Howard Stone, Laguna Hills, CA (US)

(21) Appl. No.: 18/254,272

(22) PCT Filed: Nov. 22, 2021
(86) PCT No.: PCT/US2021/060382

§ 371 Date: May 24, 2023

Related U.S. Application Data
(60) Provisional application No. 63/118,204, filed on Nov. 25, 2020.

(51) Int. Cl. A61M 1/36; A61M 60/109; A61M 60/279; A61M 60/36

(52) U.S. Cl. CPC A61M 1/3679; A61M 60/109; A61M 60/279; A61M 60/36

(57) ABSTRACT

A blood processing system includes a collection line for carrying blood, a blood separation device for separating the blood into first and second fractions, a plasma delivery line for delivering the first blood fraction to an adsorption device, and a peristaltic pump. The adsorption device adsorbs plasma of the first blood fraction. Portions of the collection and plasma delivery lines are positioned in a channel of the peristaltic pump, and the peristaltic pump causes the blood to flow through the collection line at a first flow rate and causes the first blood fraction to flow through the plasma delivery line at a second flow rate that is less than the first flow rate. In some implementations, the collection and plasma delivery lines are tubes having different internal diameters facilitating such differential flow rates. In some implementations, the peristaltic pump is the only pump in the blood processing system.

### INCORPORATION BY REFERENCE TO ANY PRIORITY APPLICATIONS

This application claims the benefit of priority under 35 U.S.C. § 119(e) to U.S. Provisional Application No. 63/118,204, filed on Nov. 25, 2020, titled “Tubing System for Delivering Blood Fractions Using Two Separate and Coordinated Volumes on a Single Pump”, which is hereby incorporated by reference herein in its entirety. Any and all applications for which a foreign or domestic priority claim is identified in the Application Data Sheet as filed with the present application are hereby incorporated by reference under 37 CFR 1.57.

### TECHNICAL FIELD

The present disclosure generally relates to blood processing systems, methods, and devices. More specifically, the present disclosure relates to systems, methods, and devices for collection of blood, separation of plasma from the collected blood, and extracorporeal processing of the plasma via adsorption.

### BACKGROUND

Various blood treatment systems and methods exist for collecting blood from a subject, processing the collected blood, and delivering the blood back to the subject. In certain scenarios, plasma or a portion of plasma from the collected blood is separated from other components of the blood, and the separated plasma is processed via dialysis, filtration, or adsorption prior to being returned to the subject. Processing the separated plasma often involves removing components therefrom. Some procedures involve processing the separated plasma to remove low-density lipoproteins, Lipoprotein (a), fibrinogen, fibrin, among other substances, for example, for subjects suffering from high blood cholesterol microcirculation issues or ischemic tissue damage. Some procedures involve removing undesirable or harmful substances, such as pathogenic antibodies, from blood plasma via immunoadsorption procedures, for example, to provide redress in relation to metabolic and immune disorders.

### SUMMARY

In many cases, it is desirable or necessary to perform plasma separation and adsorption with a blood separation device and an adsorption device. Such blood separation device can separate (all or a portion of) plasma from blood collected from a subject (via a blood collection line) and such adsorption device can act upon (all or a portion of plasma) received from the blood separation device via a plasma delivery line connected between the blood separation device and the adsorption device. Existing systems and methods for performing such plasma separation and adsorption employ separate, distinct pumps for pumping the collected blood to/through the blood separation device (via the blood collection line) and to/through the adsorption device (via the plasma delivery line). Such separate pumps must be operated in tandem (“slaved” together) such that the flow rate of blood to/through the adsorption device is coordinated, for example, to maintain a lower flow rate of plasma to the adsorption device (via the plasma delivery line) than to the blood separation device (via the blood collection line). The coordination of such separate pumps often must be controlled by complicated software and/or hardware components (for example, to detect input and/or output delivery of the pumps) and operational changes to one of such separate pumps requires coordination with each pump. Such coordinated, multi-pump systems utilized for plasma separation and adsorption purposes are not present in many hospitals and/or are too costly to make utilization practical.

Various systems, methods, and devices are disclosed herein that address one or more of the above-stated problems, among others. Some implementations of the systems disclosed herein utilize a single pump (for example, a peristaltic pump) for delivering coordinated separate volumes through separate blood lines (for example, tubes) to a blood separation device and to an adsorption device. In some implementations, a blood collection line (delivering blood to the blood separation device) and a plasma delivery line (delivering plasma from the blood separation device to the adsorption device) are both arranged within a channel of a single peristaltic pump, and one or more rollers of a rotor of the pump act to cause such separated, coordinated volumes to the blood separation and adsorption devices. In some implementations, such blood collection line and plasma delivery line have different cross-sectional areas (for example, different internal tube diameters) so as to facilitate different flow rates therethrough and to the blood separation and adsorption devices. In some implementations: the blood collection line (for example, a portion of the blood collection line arranged in the channel of the peristaltic pump) comprises a tube having an internal diameter; the plasma delivery line (for example, a portion of the plasma delivery line arranged in the channel of the peristaltic pump) comprises a tube having an internal diameter that is smaller than the internal diameter of the tube of the blood collection line, such that the flow rate through the plasma delivery line is less than the flow rate through the blood collection line. In some implementations, operating characteristics of the pump can be changed (for example, a speed of the pump) and the volumes and/or flow rates to the blood separation and adsorption devices (and/or ratios between such volumes and/or flow rates) can be varied easily in a simultaneous, coordinated manner. In some implementations, such a system can be advantageously implemented in a hospital or other environment utilizing a single pump head of a dialysis machine.

Disclosed herein is a blood processing system comprising: a blood collection line configured to carry blood; a blood separation device connected to the blood collection line and configured to separate the blood into a first fraction and a second fraction, said first fraction comprising plasma and said second fraction comprising one or more cellular components of the blood; a plasma delivery line connected to the blood separation device and configured to receive the first fraction of the blood; a plasma processing device connected to the plasma delivery line, the plasma processing device configured to process the first fraction of the blood; a pump, wherein a portion of the blood collection line and a portion of the plasma delivery line are positioned within the pump, and wherein the pump is configured to simultaneously cause the blood to flow through the blood collection line to the blood separation device and cause the first fraction of the blood to flow through the plasma delivery line to the plasma processing device; a first return line connected to the plasma processing device and configured to receive the processed the first fraction of the blood; and a second return line connected to the blood separation device and configured to receive the second fraction of the blood, wherein the second return line is connected to the first return line, thereby allowing the second fraction of the blood to be combined with the processed the first fraction of the blood.

In some implementations, said pump is the only pump in the blood processing system. In some implementations, said pump is a peristaltic pump. In some implementations, said peristaltic pump comprises a channel and a rotor, said portion of the blood collection line and said portion of the plasma delivery line are arranged within the channel, and the rotor is configured to cause the received blood to flow through the blood collection line and cause the first portion of the blood to flow through the plasma delivery line. In some implementations, said rotor comprises one or more rollers configured to compress portions of the blood collection and plasma delivery lines during rotation of the rotor. In some implementations, each of the one or more rollers are configured to simultaneously compress said portions of the blood collection and plasma delivery lines during rotation of the rotor. In some implementations, said rotor comprises at least two rollers.

In some implementations, each of said portion of the blood collection line and said portion of the plasma delivery line is arranged in a generally U-shape within said channel. In some implementations, at least a portion of said channel of said peristaltic pump comprises a generally U-shape. In some implementations, said channel is the only channel of said peristaltic pump. In some implementations, said portion of the blood collection line and said portion of the plasma delivery line are arranged to contact one another within the channel.

In some implementations: said portion of the blood collection line arranged within said channel comprises a first tube having a first internal diameter; said portion of the plasma delivery line arranged within said channel comprises a second tube having a second internal diameter; said first and second internal diameters are different; and said peristaltic pump is configured to simultaneously cause the blood flowing through the blood collection line to flow at a first flow rate and cause the first fraction of the blood flowing through the plasma delivery line to flow at a second flow rate that is different than the first flow rate. In some implementations, the second flow rate is less than the first flow rate. In some implementations, a ratio between the first internal diameter and the second internal diameter is between approximately 1 and approximately 3. In some implementations, the ratio between the first internal diameter and the second internal diameter is approximately 2. In some implementations, the second flow rate is between approximately 20% and approximately 30% of the first flow rate. In some implementations, the second flow rate is no greater than approximately 100 mL/minute. In some implementations, the first flow rate is at least approximately 50 mL/minute.

In some implementations, the blood separation device comprises: an inlet port connected to the blood collection line; a first outlet port connected to the plasma delivery line; and a second outlet port connected to the second return line. In some implementations, the blood processing system further comprises a third return line connected to both of the first and second return lines, wherein the third return line is configured to deliver the second fraction of the blood to the subject along with the processed first fraction of the blood. In some implementations, the blood processing system further comprises a branched connector connecting the first, second, and third return lines together. In some implementations, the blood processing system further comprises a blood source access device configured to connect to the third return line and deliver the second fraction of the blood to the subject along with the processed first fraction of the blood. In some implementations, said blood source access device comprises dual lumen catheter, wherein the blood collection line is configured to connect to a first port of the dual lumen catheter and wherein the third return line is configured to connect to a second port of the dual lumen catheter.

In some implementations, the blood processing system further comprises a controller in communication with the pump and configured to change one or more operating characteristics of the pump. In some implementations, said one or more operating characteristics comprises a speed of the pump. In some implementations, the pump is a peristaltic pump comprising a rotor, and wherein said one or more operating characteristics comprises a rotational speed of the rotor of the peristaltic pump.

In some implementations, the blood processing system further comprises a first pressure sensor configured to detect one or more pressure values of the first fraction prior to entering the plasma processing device, wherein the controller is configured to receive said one or more pressure values and change said one or more operating characteristics of the pump based upon said one or more pressure values. In some implementations, the controller is further configured to compare said one or more pressure values to one or more thresholds and change said one or more operating characteristics of the pump based upon said comparison. In some implementations, the blood processing system further comprises a second pressure sensor configured to detect one or more pressure values of the blood prior to entering the blood separation device, wherein the controller is configured to receive said one or more pressure values from the second pressure sensor and change said one or more operating characteristics of the pump based upon said one or more pressure values received from said second pressure and based upon said one or more pressure values received from said first pressure sensor. In some implementations, the controller is further configured to compare said one or more pressure values received from the second pressure sensor to one or more thresholds and change said one or more operating characteristics of the pump based upon said comparison. In some implementations, the controller is further configured to compare said one or more pressure values received from said first pressure sensor with said one or more pressure values received from said second pressure sensor and change said one or more operating characteristics of the pump based upon said comparison.

In some implementations, the blood separation device comprises a centrifugal separator. In some implementations, the blood separation device comprises one or more membranes. In some implementations, each of the one or more membranes comprises a pore size between approximately 0.2 μm and approximately 1 μm.

In some implementations, said first fraction comprises a first percentage of a total volume of the blood entering the blood separation device and said second fraction comprises a second percentage of said total volume. In some implementations, said first percentage is less than said second percentage. In some implementations, said first percentage is between approximately 5% and approximately 50% of said total volume. In some implementations, said first percentage is between approximately 20% and approximately 30% of said total volume.

In some implementations, said second fraction of the blood comprises plasma and said one or more cellular components. In some implementations, substantially an entirety of said first fraction comprises plasma. In some implementations, said one or more cellular components comprises at least one of white blood cells, red blood cells, and platelets. In some implementations, said blood collection line comprises one or more tubes. In some implementations, said blood collection line comprises a single tube. In some implementations, said plasma delivery line comprises one or more tubes. In some implementations, said plasma delivery line comprises a single tube.

In some implementations, said plasma processing device is an adsorption device configured to adsorb at least a portion of the plasma of the first fraction of the blood to remove one or more substances from the first fraction of the blood. In some implementations, said adsorption device is in the form of a cartridge or column.

In some implementations, the adsorption device comprises one or more absorptive materials selected from the group consisting of thermally expanded graphite (TEG), graphene nanoplatelets (GNPs), polymer derived ceramic carbide-derived carbon (PDC-CDC), an ion exchange resin, and a non-ion exchange resin.

In some implementations, said blood collection line is configured to receive blood from a blood source. In some implementations, said blood source is a container. In some implementations, the blood processing system further comprises a blood source access device connected to the blood collection line, wherein said blood source comprises one of a vein, an artery, or an arteriovenous fistula of a subject, and wherein said blood source access device is configured to withdraw said blood from the subject's vein, artery, or arteriovenous fistula.

In some implementations, said pump is the only pump arranged along the blood collection line and the plasma delivery line. In some implementations, said pump is the only pump arranged between the blood separation device and the plasma processing device.

Disclosed herein is a blood processing system comprising: a blood collection line; a blood separation device; a plasma delivery line; an adsorption device; a peristaltic pump; a first return line; and a second return line. The blood collection line can be configured to carry blood. The blood separation device can comprise an inlet port connected to the blood collection line, a first outlet port, and a second outlet port. The blood separation device can be configured to: receive the blood from the blood collection line via the inlet port; separate the blood into a first fraction and a second fraction, said first fraction comprising plasma and said second fraction comprising one or more cellular components of the blood; direct the first fraction of the blood to the first outlet port; and direct the second fraction of the blood to the second outlet port. The plasma delivery line can be connected to the first outlet port of the blood separation device and configured to receive the first fraction of the blood. The adsorption device can comprise an inlet port connected to the plasma delivery line and an outlet port. The adsorption device can adsorb at least a portion of the plasma of the first fraction of the blood to remove one or more substances from the first fraction of the blood. The peristaltic pump can comprise a channel and a rotor. A portion of the blood collection line comprising a first internal diameter and a portion of the plasma delivery line comprising a second internal diameter can be arranged adjacent one another within the channel. The first and second internal diameters can be different from one another. The rotor can be configured to cause the blood to flow through the blood collection line to the blood separation device at a first flow rate and cause the first portion of the blood to flow through the plasma delivery line to the adsorption device at a second flow rate that is less than the first flow rate. The first return line can be connected to the outlet of the adsorption device and can be configured to receive the processed the first fraction of the blood. The second return line can be connected to second outlet port of the blood separation device and configured to receive the second fraction of the blood, wherein the second return line connected to the first return line, thereby allowing the second fraction of the blood to be combined with the processed the first fraction of the blood.

In some implementations, the rotor is configured to simultaneously cause the blood to flow through the blood collection line to the blood separation device at the first flow rate and the first portion of the blood to flow through the plasma delivery line to the adsorption device at the second flow rate. In some implementations, said peristaltic pump is the only pump in the blood processing system. In some implementations, said peristaltic pump is the only pump arranged along the blood collection line and the plasma delivery line. In some implementations, said peristaltic pump is the only pump arranged between the blood separation device and the plasma processing device.

In some implementations, said rotor is configured to rotate and comprises one or more rollers configured to compress said portions of the blood collection and plasma delivery lines during rotation of the rotor. In some implementations, each of the one or more rollers are configured to simultaneously compress said portions of the blood collection and plasma delivery lines during rotation of the rotor. In some implementations, said rotor comprises at least two rollers.

In some implementations, each of said portion of the blood collection line and said portion of the plasma delivery line is arranged in a generally U-shape within said channel. In some implementations, at least a portion of said channel of said peristaltic pump comprises a generally U-shape. In some implementations, said channel is the only channel of said peristaltic pump. In some implementations, said portion of the blood collection line and said portion of the plasma delivery line are arranged to contact one another within the channel.

In some implementations, said portion of the blood collection line and said portion of the plasma delivery line each comprise a tube. In some implementations, a ratio between the first internal diameter and the second internal diameter is between approximately 1 and approximately 3. In some implementations, the ratio between the first internal diameter and the second internal diameter is approximately 2. In some implementations, the second flow rate is between approximately 20% and approximately 30% of the first flow rate. In some implementations, the second flow rate is no greater than approximately 100 mL/minute. In some implementations, the first flow rate is at least 50 mL/minute.

In some implementations, the blood processing system further comprising a controller in communication with the peristaltic pump and configured to change one or more operating characteristics of the peristaltic pump. In some implementations, the blood separation device comprises one or more membranes. In some implementations, each of the one or more membranes comprises a pore size between approximately 0.2 μm and approximately 1 μm.

In some implementations, said first fraction comprises a first percentage of a total volume of the blood entering the blood separation device and said second fraction comprises a second percentage of said total volume, and wherein said first percentage is less than said second percentage. In some implementations, said first percentage is between approximately 20% and approximately 30% of said total volume. In some implementations, each of said blood collection line and said plasma delivery line comprises a single tube.

In some implementations, said adsorption device is in the form of a cartridge or column. In some implementations, the adsorption device comprises one or more absorptive materials selected from the group consisting of thermally expanded graphite (TEG), graphene nanoplatelets (GNPs), polymer derived ceramic carbide-derived carbon (PDC-CDC), an ion exchange resin, and a non-ion exchange resin.

Disclosed herein is a blood processing system comprising: a first blood transport line configured to carry blood to a blood separation device, the blood separation device configured to separate the blood into a first fraction and a second fraction, said first fraction comprising plasma and said second fraction comprising one or more cellular components of the blood; a second blood transport line connected to the blood separation device and configured to deliver the first fraction of the blood to an adsorption device, the adsorption device configured to adsorb at least a portion of the plasma of the first fraction of the blood to remove one or more substances from the first fraction of the blood; and a peristaltic pump configured to simultaneously cause the blood to flow through the first blood transport line to the blood separation device and the first fraction of the blood to flow through the second blood transport line to the adsorption device.

In some implementations, said peristaltic pump is the only pump in the blood processing system. In some implementations, no other pumps cause the blood to flow through the first blood transport line to the blood separation device and the first fraction of the blood to flow through the second blood transport line to the adsorption device.

In some implementations, a portion of the first blood transport line and a portion of the second blood transport line are positioned within the peristaltic pump. In some implementations, the peristaltic pump comprises a channel and said portions of the first and second blood transport lines are positioned within said channel. In some implementations, said peristaltic pump further comprises a rotor, said rotor configured to rotate and comprising one or more rollers, said one or more rollers configured to compress said portions of the first and second blood transport lines during rotation of the rotor. In some implementations, each of the one or more rollers are configured to simultaneously compress said portions of the first and second blood transport lines during rotation of the rotor. In some implementations, said rotor comprises at least two rollers.

In some implementations, each of said portions of the first and second blood transport lines is arranged in a generally U-shape within said channel. In some implementations, said channel is the only channel in said peristaltic pump. In some implementations, said channel comprises a generally U-shape. In some implementations, each of said portions of the first and second blood transport lines comprises a tube.

In some implementations, said portion of the first blood transport line comprises a tube having a first internal diameter and said portion of the second transport line comprises a tube having a second internal diameter that is less than the first internal diameter. In some implementations, a ratio between the first internal diameter and the second internal diameter is between approximately 1 and approximately 3. In some implementations, a ratio between the first internal diameter and the second internal diameter is approximately 2.

In some implementations, said peristaltic pump is configured to simultaneously cause the blood to flow through the first blood transport line to the blood separation device at a first flow rate and the first fraction of the blood to flow through the second blood transport line to the adsorption device at a second flow rate that is less than the first flow rate. In some implementations, the second flow rate is between approximately 20% and approximately 30% of the first flow rate.

Disclosed herein is a method of processing blood comprising: carrying blood with a blood collection line; separating the blood into a first fraction and a second fraction with a blood separation device, said first fraction comprising plasma and said second fraction comprising one or more cellular components of the blood; delivering the first fraction of the blood to a plasma processing device with a plasma delivery line; simultaneously pumping, with a single pump, the blood through the blood collection line to the blood separation device and the first fraction of the blood to the plasma processing device, wherein a portion of the blood collection line and a portion of the plasma delivery line are positioned within the pump; processing the first fraction of the blood with the plasma processing device; and combining said second fraction of the blood with the processed first fraction of the blood.

In some implementations, the method further comprises receiving, from a blood source access device, the blood with the blood collection line. In some implementations, said receiving, from the blood source access device, the blood with the blood collection line comprises receiving the blood when the blood source access device is secured to a subject.

In some implementations, the method further comprises delivering said second fraction of the blood with the processed first fraction of the blood to a subject.

In some implementations, the method further comprises: carrying the processed the first fraction of the blood through a first return line after the processed first fraction of the blood exits the plasma processing device; and carrying the second fraction of the blood through a second return line after the second fraction exits the blood separation device. In some implementations, said combining said second fraction of the blood with the processed first fraction of the blood comprises: delivering the processed first fraction of the blood through the first return line to a third return line; and delivering the second fraction of the blood through the second return line to the third return line. In some implementations, the third return line is connected to the first and second return lines.

In some implementations, the method further comprises delivering the combined second fraction of the blood and the processed first fraction of the blood to a subject with a blood source access device. In some implementations, said combining said second fraction of the blood with the processed first fraction of the blood further comprises allowing said second fraction of the blood and the processed first fraction of the blood to flow through a branched connector connected to the first, second, and third return lines.

In some implementations, said simultaneously pumping with the single pump further comprises simultaneously causing: the blood flowing through the blood collection line to flow at a first flow rate; and the first fraction of the blood flowing through the plasma delivery line to flow at a second flow rate that is different than the first flow rate. In some implementations, the second flow rate is less than the first flow rate. In some implementations, the second flow rate is between approximately 20% and approximately 30% of the first flow rate. In some implementations, the second flow rate is no greater than 100 mL/minute. In some implementations, the first flow rate is at least 50 mL/minute.

In some implementations: said pump is a peristaltic pump comprising a channel and a rotor; the method further comprises positioning the portion of the blood collection line and the portion of the plasma delivery line within the channel; and said simultaneously pumping comprises rotating the rotor to cause the blood to flow through the blood collection line and cause the first fraction of the blood to flow through the plasma delivery line to the plasma purification device. In some implementations, the rotor comprises one or more rollers, and wherein said simultaneously pumping further comprises causing each of the one or more rollers to simultaneously compress portions of the blood collection and plasma delivery lines during rotation of the rotor.

In some implementations, said positioning said portion of the blood collection line and said portion of the plasma delivery line within the channel comprises arranging each of said portion of the blood collection line and said portion of the plasma delivery line in a generally U-shaped configuration within the channel. In some implementations, said positioning said portion of the blood collection line and said portion of the plasma delivery line within the channel comprises bending said portion of the blood collection line and said portion of the plasma delivery line and inserting bent portions of the blood collection and plasma delivery lines within the channel. In some implementations, said channel is the only channel of said peristaltic pump. In some implementations, said channel comprises a generally U-shape.

In some implementations, said portion of the blood collection line comprises a tube having a first internal diameter, and wherein said portion of the plasma delivery line comprises a tube having a second internal diameter that is different than the first internal diameter. In some implementations, the second internal diameter is less than the first internal diameter. In some implementations, a ratio between the first internal diameter and the second internal diameter is between approximately 1 and approximately 3.

In some implementations, the method further comprises changing one or more operating characteristics of the pump with a controller. In some implementations, said changing said one or more operating characteristics of the pump comprises changing a speed of the pump.

In some implementations, said blood separation device comprises one or more membranes, and wherein said separating said the blood into said first and second fractions comprises allowing the blood to flow through said one or more membranes. In some implementations, said first fraction comprises a first percentage of a total volume of the blood entering the blood separation device and said second fraction comprises a second percentage of said total volume. In some implementations, said first percentage is less than said second percentage. In some implementations, said first percentage is between approximately 5% and approximately 50% of said total volume. In some implementations, said first percentage is between approximately 20% and approximately 30% of said total volume. In some implementations, said second fraction of the blood comprises plasma and said one or more cellular components. In some implementations, substantially an entirety of said first fraction comprises plasma. In some implementations, said one or more cellular components comprises at least one of white blood cells, red blood cells, and platelets. In some implementations, said blood collection line comprises a single tube. In some implementations, said plasma delivery line comprises a single tube.

In some implementations, said plasma processing device is an adsorption device configured to adsorb at least a portion of the plasma of the first fraction of the blood to remove one or more substances from the first fraction of the blood. In some implementations, said adsorption device is in the form of a cartridge or column. In some implementations, the adsorption device comprises one or more absorptive materials selected from the group consisting of thermally expanded graphite (TEG), graphene nanoplatelets (GNPs), polymer derived ceramic carbide-derived carbon (PDC-CDC), an ion exchange resin, and a non-ion exchange resin.

For purposes of summarizing the disclosure, certain aspects, advantages, and novel features are discussed herein. It is to be understood that not necessarily all such aspects, advantages, or features will be embodied in any particular embodiment of the disclosure, and an artisan would recognize from the disclosure herein a myriad of combinations of such aspects, advantages, or features.

### BRIEF DESCRIPTION OF THE DRAWINGS

Certain features of this disclosure are described below with reference to the drawings. The illustrated embodiments are intended to illustrate, but not to limit, the embodiments. Various features of the different disclosed embodiments can be combined to form further embodiments, which are part of this disclosure.

FIG. 1 illustrates a schematic diagram of a blood processing system in accordance with aspects of this disclosure.

FIG. 2A illustrates a top view of a peristaltic pump in accordance with aspects of this disclosure.

FIG. 2B illustrates a top view of the peristaltic pump of FIG. 2A during operation in accordance with aspects of this disclosure.

FIG. 3 illustrates a block diagram for a method of processing blood in accordance with aspects of this disclosure.

### DETAILED DESCRIPTION

Various features and advantages of this disclosure will herein be described with reference to the accompanying figures. The following description is merely illustrative in nature and is in no way intended to limit the disclosure, its application, or uses. This disclosure extends beyond the specifically disclosed embodiments and/or uses and obvious modifications and equivalents thereof. Thus, it is intended that the scope of this disclosure should not be limited by any particular embodiments described below. The features of the illustrated embodiments can be modified, combined, removed, and/or substituted as will be apparent to those of ordinary skill in the art upon consideration of the principles disclosed herein.

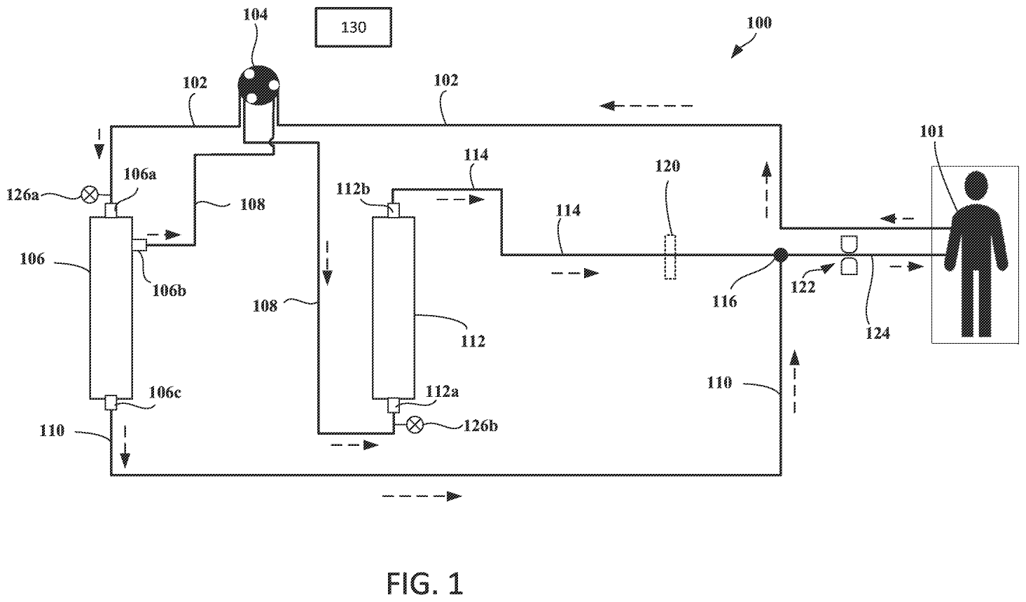

FIG. 1 illustrates a schematic diagram of a blood processing system 100. The blood processing system 100 can include a blood collection line 102, a blood separation device 106, a pump 104, a plasma processing device 112, a plasma delivery line 108 connected between the blood separation device 106 and the plasma processing device 112, a first return line 110 connected to the blood separation device 106, and a second return line 114 connected to the plasma processing device 112, among other components. The blood processing system 100 can be utilized to carry out a variety of procedures relating to blood collection, processing, and/or return to a subject 101. For example, the blood processing system 100 can be utilized in various procedures where it is desirable or necessary to process (for example, remove one or more components from) plasma of the subject's blood, as discussed further below. Subject 101 can be a patient, for example, in a hospital or other care facility. Any or all of the blood collection line 102, the blood separation device 106, the pump 104, the plasma delivery line 108, the plasma processing device 112, the first return line 110 connected to the blood separation device 106, and/or the second return line 114 can form a blood processing circuit which defines a flow path of blood therethrough, where the flow path is illustrated through such flow circuit using the dotted arrows.

The blood collection line 102 (which may be referred to as a “whole blood collection line”, a “collection line”, and/or a “first transport line”) can receive blood from a subject 101, for example, via a blood source access device that secures to a portion of the subject's body (for example, arm, leg, among other portions) and accesses blood via the subject's vasculature. Such blood source access device can be any of a variety of devices that can be utilized to withdraw blood from the subject, for example, from a vein, an artery, and/or an arteriovenous fistula in the subject's arm or leg. Such blood source access device can include a needle usable to penetrate the subject's skin and gain access to the subject's blood via a vein, artery, and/or arteriovenous fistula. Such blood source access device can be a dual lumen catheter, as another example. The blood collection line 102 can be configured to connect (for example, removably connect) to such blood source access device to receive blood from the subject and deliver the received blood to the blood separation device 106. The blood collection line 102 can include one or a plurality of tubes extending between and connected to (for example, in series) the blood source access device on subject 101 and the blood separation device 106. As shown in FIG. 1 and as discussed further below, the blood processing system 100 can include a pump 104 that causes the subject's blood to flow through the blood collection line 102 to the blood separation device 106. As also discussed further below, the blood collection line 102 (for example, a portion thereof) can be positioned inside a portion of pump 104 (for example, a channel of pump 104 where pump 104 is a peristaltic pump). In some implementations, the blood collection line 102 comprises a single tube, for example, extending between and connected to the blood source access device and the blood separation device 106. In alternative implementations, the blood collection line 102 comprises a plurality of tubes connected to one another (for example, with tube connectors) and extending between the blood source access device and the blood separation device 106.

The blood separation device 106 can connect to and receive blood from the blood collection line 102. The blood separation device 106 can include an inlet port 106a (which may also be referred to as an “inlet”), a first outlet port 106b (which may also be referred to as a “first outlet”), and a second outlet port 106b (which may also be referred to as a “second outlet”). The inlet port 106a can be configured to connect to blood collection line 102 (for example, an end of the blood collection 102). The first outlet port 106b can be configured to connect to the plasma delivery line 108 (for example, an end of the plasma delivery line 108). The second outlet port 106c can be configured to connect to the return line 110 (for example, an end of the return line 110). In some implementations, inlet port 106a is located at a first end of the blood separation device 106 and outlet port 106c is located at a second end of the blood separation device 106 that is opposite the first end of the blood separation device 106. In some implementations, outlet port 106b is located on a side of blood separation device 106, for example, between such first and second ends and/or between the inlet port 106a and the outlet port 106c. Although FIG. 1 illustrates the blood separation device 106 having inlet port 106a, outlet port 106b, and outlet port 106c, the blood separation device 106 can include additional ports, in some implementations.

The blood separation device 106 can separate the received blood (which may be referred to as “whole blood” prior to separation) into various portions, for example, to separate components of the blood. For example, the blood separation device 106 can separate the received blood into a first fraction that includes a portion of the plasma and a second fraction that includes a portion of the plasma and also one or more cellular components (for example, red blood cells, white blood cells, and/or platelets). Such “first fraction” and “second fraction” of the received blood may also be referred to as “first” and “second” “portions” of the received blood herein. Separating at least a portion of the plasma from the received blood can advantageously allow the separated plasma to be processed (for example, via adsorption) as discussed in more detail below. Such first fraction including a portion of the plasma can be directed to a plasma delivery line 108 (for example, via outlet port 106b) as discussed in more detail below. Such second fraction including a portion of the plasma and also one or more cellular components (for example, red blood cells, white blood cells, and/or platelets) can be directed to a return line 110 (for example, via outlet port 106c) as also discussed in more detail below.

The separated, first fraction of the received blood can comprise plasma representing a first percentage of a total volume of the received blood entering the blood separation device 106 and the separated, second fraction of the received blood can comprise a second percentage of such total volume. In some implementations, such first percentage includes between approximately 5% and approximately 50% of such total volume of the received blood entering the blood separation device 106, for example, between approximately 10% and approximately 45%, between approximately 15% and approximately 40%, between approximately 20% and approximately 35%, between approximately 25% and approximately 30%, or between approximately 20% and approximately 30%, or any value or range within or bounded by any of these ranges or values, although values outside these values or ranges can be used in some cases. In some implementations, such second percentage includes between approximately 50% and approximately 95% of such total volume of the received blood entering the blood separation device 106, for example, between approximately 55% and approximately 90%, between approximately 60% and approximately 85%, between approximately 65% and approximately 80%, between approximately 70% and approximately 75%, or between approximately 70% and approximately 80%, or any value or range within or bounded by any of these ranges or values, although values outside these values or ranges can be used in some cases. In some implementations, the first percentage is smaller than such second percentage.

In some implementations, the blood separation device 106 comprises one or more membranes that can be utilized to separate the received blood, for example, into the first and second fractions discussed above, in order to separate a portion of the plasma from the received blood. The one or more membranes can include a variety of pore sizes to accomplish such separation. In some implementations, the one or more membranes have a pore size that is between approximately 0.1 μm and approximately 1.5 μm, for example, between approximately 0.2 μm and approximately 1.4 μm, between approximately 0.3 μm and approximately 1.3 μm, between approximately 0.4 μm and approximately 1.2 μm, between approximately 0.5 μm and approximately 1.1 μm, between approximately 0.6 μm and approximately 1 μm, between approximately 0.7 μm and approximately 0.9 μm, between approximately 0.2 μm and approximately 1 μm, between approximately 0.3 μm and approximately 0.9 μm, between approximately 0.4 μm and approximately 0.8 μm, or between approximately 0.5 μm and approximately 0.7 μm. In some implementations, the one or more membranes comprise polyethersulfone. The blood separation device 106 can be any of the plasma filters manufactured by Medica SPA, for example, the PLASMART Versatile®-PES plasma filters. In some variants, the blood separation device 106 comprise a centrifuge usable to centrifugally separate blood components.

As discussed above, the blood processing system 100 can include a plasma processing device 112. The plasma processing device 112 (which may also be referred to herein as “plasma purification device”) can be any device capable of processing plasma received from the blood separation device 106 (for example, via plasma delivery line 108). Such processing can involve any of a variety of techniques for removing one or more substances (which may also be referred to as “components” herein) from the received plasma. For example, the plasma processing device 112 can be configured to filter the plasma and/or adsorb at least a portion of the received plasma to remove one or more substances therefrom. In some implementations, the plasma processing device 112 is an adsorption device comprising one or more adsorptive materials suitable for removing one or more components from the plasma, a technique sometimes referred to as hemoperfusion or plasma perfusion. In such implementations, the plasma processing device 112 can advantageously be utilized to remove specific components to provide effective therapeutic intervention in relation to treatment of atherosclerosis, cancer, degenerative and/or autoimmune diseases, among others. In such implementations, the plasma processing device 112 can be utilized to remove cytokines from blood, for example, for medical conditions causing an overactivation of inflammatory response and release of dangerously high levels of pro- and anti-inflammatory cytokines, and which may be associated with sepsis among other conditions. The plasma processing device 112 can include one or a plurality of absorbents, including but not limited to polymer-derived carbons, carbide-derived carbons, polymer adsorbents (polystyrene and cellulose), modified inorganic materials, and/or graphene-based materials (such as expanded graphite (EGr), graphene nanoplatelets (GnP), and/or granulated graphene nanoplatelets (GnP:PTFE)), among other materials. As another example, the plasma processing device 112 can include one or a plurality of absorbents, including but not limited to thermally expanded graphite (TEG), graphene nanoplatelets (GNPs), polymer derived ceramic carbide-derived carbon (PDC-CDC), an ion exchange resin, and/or a non-ion exchange resin. As another example, the plasma processing device 112 can include any of the adsorbents discussed in Mykola Seredych et al, “Graphene-Based Materials for the Fast Removal of Cytokines from Blood Plasma”, ACS Appl. Bio Mater. 2018, 1, 436-443, which is hereby incorporated by reference herein in its entirety. As another example, the plasma processing device 112 can be configured to remove proteins, such as cytokines, using any of the methods, systems, and/or devices described in U.S. Pat. No. 11,123,465, titled “Methods of Using Thermally Expanded Graphite to Remove Proteins from Blood and to Treat Sepsis”, issued on Sep. 21, 2021, which is hereby incorporated by reference herein in its entirety. The plasma processing device 112 can include an inlet port 112a and an outlet port 112b for connecting to the return line 114. The inlet and outlet ports 112a, 112b can be located on opposite ends of the plasma processing device 112 in some implementations. In some implementations, the plasma processing device 112 is in the form of a cartridge.

As shown in FIG. 1, the plasma processing device 112 can receive plasma from the blood separation device 106 via the plasma delivery line 108 (which may also be referred to as a “second transport line”). Plasma delivery line 108 may also be referred to as a “pre-adsorption line,” for example, when plasma processing device 112 is an adsorption device. As discussed above, the blood separation device 106 can separate the received blood (which may be referred to as “whole blood” prior to separation by blood separation device 106) into various portions, such as a first fraction that includes a portion of the plasma from the received blood and a second fraction that includes a portion of the plasma and also one or more cellular components (for example, red blood cells, white blood cells, and/or platelets). The plasma delivery line 108 can connect to and receive such first fraction from the outlet port 106b of the blood separation device 106. The plasma delivery line 108 can connect and deliver such first fraction to the plasma processing device 112. For example, the plasma delivery line 112 can connect to the inlet port 112a of the plasma processing device 112. In some implementations, the plasma delivery line 112 comprises a single tube, for example, extending between and connected to the blood separation device 106 and the plasma processing device 112. In alternative implementations, the plasma delivery line 112 comprises a plurality of tubes connected to one another (for example, with tube connectors) and extending between the blood separation device 106 and the plasma processing device 112.

As shown, the blood processing system 100 can include a pump 104 to facilitate movement of blood (and/or portions thereof) through the flow circuit formed by various components of the blood processing system 100. FIG. 1 illustrates pump 104 along the blood collection line 102. The pump 104 can be positioned along the blood collection line 102 between ends of the blood collection line 102 that connect to the blood separation device 106 and to the subject 101 (for example, via a blood source access device as discussed above). Described another way, a portion of the blood collection line 102 between such ends can be arranged to engage (and/or be engaged by) pump 104. FIG. 1 also illustrates pump 104 along the plasma delivery line 108, and pump 104 can be positioned along the plasma delivery line 108 between ends of the plasma delivery line 104 that connect to the blood separation device 106 (for example, via outlet port 106b) and the plasma processing device 112 (for example, via inlet port 112a). Described another way, a portion of the plasma delivery line 104 between ends of the plasma delivery line 104 can be arranged to engage (and/or be engaged by) pump 104. Such implementations can allow the pump 104 to cause the received blood to flow towards the blood separation device 106 in the flow direction illustrated in dotted arrow in FIG. 1.

As discussed above, the blood processing system 100 can include the plasma processing device 112, which can be an adsorption device as discussed above, that receives at least a portion of plasma separated from the received blood by the blood separation device 106 via the plasma delivery line 108. Existing blood processing systems having a blood collection line, blood separation device, plasma delivery line, and plasma processing device include two distinct, separate pumps—one for pumping blood through the blood collection line to the blood separation device and a separate pump for pumping separated plasma from the blood separation device through the plasma delivery line to the plasma processing device 112. Such arrangement allows for the flow rate to the plasma processing device to be less than the flow rate blood separation device, which is an important consideration in blood processing systems including such components given flow rate limitations to plasma processing devices (such as adsorption devices). However, in such conventional systems, such separate pumps must be operated in tandem (“slaved” together) such that the flow rate of blood to/through the plasma processing device is coordinated. The coordination of such separate pumps often must be controlled by complicated software and/or hardware components (for example, to detect input and/or output delivery of the pumps) and operational changes to one of such separate pumps requires coordination with each pump. For example, flow volume changes through either of such multi-pump systems requires software component(s) and/or sensor(s) to detect input and output characteristics of each pump for system coordination. Such coordinated, multi-pump systems utilized for plasma separation and processing purposes are not present in many hospitals and/or are too costly to make utilization practical.

Advantageously, in contrast to the above-described existing blood processing systems, blood processing system 100 can include a single pump 104 for causing blood to flow through blood collection line 102 (to blood separation device 106) and causing separated plasma to flow through plasma delivery line 108 (to plasma processing device 112). As illustrated in FIG. 1, such pump 104 can be arranged along both of blood collection line 102 and plasma delivery line 104. In such arrangement, pump 104 can simultaneously pump blood through blood collection line 102 and separated plasma through plasma delivery line 108, and operational changes to pump 104 (for example, to change pump speed) can simultaneously change flow rate and/or flow volumes through blood collection line 102 (to blood separation device 106) and through plasma delivery line 108 (to plasma processing device 112).

To facilitate differential flow rates through blood collection line 102 (to blood separation device 106) and through plasma delivery line 108 (to plasma processing device 112), blood collection line 102 (or a portion thereof) and plasma delivery line 108 (or a portion thereof) can have different cross-sectional areas. For example, where blood collection line 102 and plasma delivery line 108 each comprise a tube having a circular cross-section, an internal diameter of the tube of the blood collection line 102 can be larger than an internal diameter of the tube of the plasma delivery line 108. Such configurations allow the flow rate to the plasma processing device 112 to be less than the flow rate to the blood separation device 106. In some implementations, pump 104 is the only pump in blood processing system 100. In some implementations, pump 104 is the only pump in blood processing system 100 that causes blood to flow to the blood separation device 106 (for example, through the blood collection line 102) and separated plasma to flow to the plasma processing device 112 (for example, through the plasma delivery line 108). In some implementations, pump 104 is the only pump in blood processing system 100 arranged along a flow path between a subject 101 and the blood separation device 106 and arranged along a flow path between the blood separation device 106 and the plasma processing device 112.

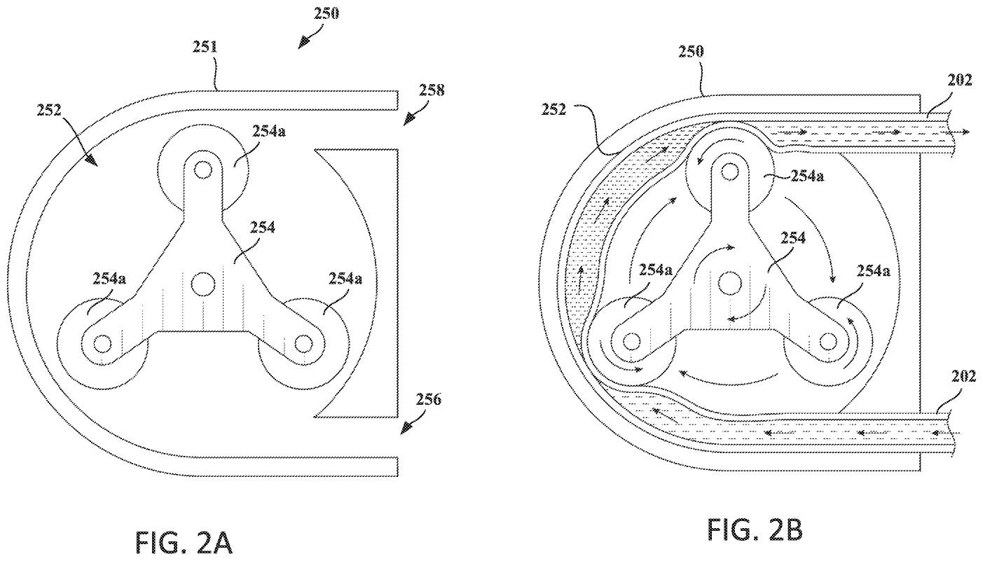

The pump 104 can be a peristaltic pump, for example, similar or identical to the illustrative peristaltic pump 250 shown in FIG. 2A. With reference to FIG. 2A, peristaltic pump 250 can include a housing 251 (which may also be referred to as a “body”) and a channel 252 (which may also be referred to as a “raceway”). The channel 252 can be positioned within and/or defined by an interior of the housing 251. The channel 252 can at least partially form and/or define a generally U-shape, for example, to facilitate arrangement of portions of the blood collection and plasma delivery lines 102, 108 arranged in a generally U-shape when positioned within the channel 252 as discussed further below. The peristaltic pump 250 can include an entrance 256 into channel 252 and an exit 258 from channel 252. The entrance 256 and the exit 258 can allow a tube to enter and exit the channel 252. The peristaltic pump 250 can include a rotor 254. The rotor 254 can be configured for rotational movement within housing 251 (for example, within an interior of housing 251). The rotor 254 can be configured to rotate about a central axis of the peristaltic pump 252, housing 251, and/or rotor 254. The rotor 254 can include one or more rollers (for example, one, two, three, four, five, six or more rollers). In the illustrated implementation of FIG. 2A, rotor 254 includes three rollers 254a, however, an alternative number of rollers is possible (for example, two rollers). Each of the rollers 254a can be connected to portions of the rotor 254 (for example, near a perimeter of the rotor 254) and can be configured to rotate relative to central axes thereof. The rollers 254a can act to compress portions of the blood collection and plasma delivery lines 102, 108 during rotation of the rotor 254 as discussed further below.

FIG. 2B illustrates a tube 202 positioned within channel 252 of the peristaltic pump 250. FIG. 2B also illustrates operation of peristaltic pump 250, whereby rotation of the rotor 254 causes rollers 254a to compress portions of the tube 202, thereby causing fluid (blood) to flow in the direction indicated by arrows (for example, in a direction from the entrance 256 to the exit 258 along channel 252 and a portion of a length of the tube 202). As mentioned previously and as illustrated in FIG. 1, the pump 104 can be utilized to cause blood to flow through the blood collection line 102 and the plasma delivery line 108. Where the pump 104 is similar or identical to peristaltic pump 250, the blood collection line 102 (for example, a portion thereof) and the plasma delivery line 108 (for example, a portion thereof) can be arranged within the channel 252 in a similar or identical manner as that illustrated with respect to tube 202. For example, in some implementations, a portion of the blood collection 102 can be positioned within channel 252 and a portion of the plasma delivery line 108 can also be positioned within channel 252, for example, adjacent to and/or contacting the portion of the blood collection line 102 within channel 252. In such arrangements, rotor 254 (for example, rollers 254a) can simultaneously compress such portions of the blood collection and plasma delivery lines 102, 108 to cause flow through the blood collection and plasma delivery lines 102, 108.

In some implementations, peristaltic pump 250 comprises only one channel—for example, channel 252—and such portions of the blood collection and plasma delivery lines 102, 108 are both positioned within channel 252. In some implementations, such portions of the blood collection and plasma delivery lines 102, 108 are arranged in a generally U-shape within channel 252. In some implementations, the peristaltic pump 252 is configured to allow the blood collection and plasma delivery lines 102, 108 are both positioned within channel 252 to extend through entrance 256, channel 252, and/or exit 258 uninterrupted. For example, in some implementations, peristaltic pump 250 does not include tube(s) and/or does not include connectors that require connection to blood collection and plasma delivery lines 102, 108 (for example, which would require blood collection and plasma delivery lines 102, 108 to comprise separate tubes, each connected to a connector of pump 250 at the entrance 256 or exit 258). Peristaltic pump 250 may, for example, be configured to allow blood collection and plasma delivery lines 102, 108 to be removably mounted within channel 252. However, in some variants, peristaltic pump 250 includes connectors at or near each of entrance 256 and exit 258 which can connect to ends of portions of blood collection and plasma delivery lines 102. In some variants, peristaltic pump 250 can include, for example, two tubes, each extending within channel 252 and between such ends of the portions of the blood collection and plasma delivery lines 102, 108 so that the rotor 252 and rollers 254a can cause flow through the same (for example, whereby rollers 254a can compress portions of such two tubes). In such variants, each of such two tubes in peristaltic pump 250 are separate from blood collection and plasma delivery lines 102, 108 and can comprise different cross-sectional areas (for example, different internal diameters) that facilitate different flow rates therethrough and through the respectively connected ends of the portions of the blood collection and plasma delivery lines 102, 108.

As discussed previously, the blood collection line 102 (or a portion thereof) and the plasma delivery line 108 (or a portion thereof) can have different cross-sectional areas to facilitate differential flow rates through blood collection line 102 (to blood separation device 106) and through plasma delivery line 108 (to plasma processing device 112). As also discussed above, portions of the blood collection and plasma delivery lines 102, 108 can be positioned within a channel of a peristaltic pump in a manner similar or identical to that shown with respect to tube 202 in channel 252 of peristaltic pump 250. Such configurations allow the flow rate through the plasma delivery line 108 and to the plasma processing device 112 to be less than the flow rate through the blood collection line 102 and to the blood separation device 106.

In some implementations, the blood collection line 102 (for example, a portion of the blood collection line 102 positioned within channel 252 of peristaltic pump 250) comprises a tube having an internal cross-sectional area (defining a flow area therethrough) that is larger than an internal cross-sectional area of a tube comprised by the plasma delivery line 108 (for example, a portion of the plasma delivery line 108 positioned within channel 252 of peristaltic pump 250). Such tube of the blood collection line 102 can comprise an internal cross-sectional area that is between approximately 1 and approximately 5 times larger than an internal cross-sectional area of the tube of the plasma delivery line 108 (for example, between approximately 1 and approximately 4, between approximately 1 and approximately 3, between approximately 1.5 and approximately 2.5, or approximately 2 times larger than an internal cross-sectional area of the such tube of the plasma delivery line 108, or any value or range within or bounded by any of these ranges or values, although values outside these values or ranges can be used in some cases).

In some implementations, the blood collection line 102 (for example, a portion of the blood collection line 102 positioned within channel 252 of peristaltic pump 250) and the plasma delivery line 108 (for example, a portion of the plasma delivery line 108 positioned within channel 252 of peristaltic pump 250) each comprise a tube having a circular cross-section. In some implementations, the blood collection line 102 (for example, a portion of the blood collection line 102 positioned within channel 252 of peristaltic pump 250) comprises a tube having an internal diameter that is larger than an internal diameter of a tube comprised by the plasma delivery line 108 (for example, a portion of the plasma delivery line 108 positioned within channel 252 of peristaltic pump 250). Such tube of the blood collection line 102 can comprise an internal diameter that is between approximately 1 and approximately 5 times larger than an internal diameter of such tube of the plasma delivery line 108 (for example, between approximately 1 and approximately 4, between approximately 1 and approximately 3, between approximately 1.5 and approximately 2.5, or approximately 2 times larger than an internal diameter of the such tube of the plasma delivery line 108, or any value or range within or bounded by any of these ranges or values, although values outside these values or ranges can be used in some cases).

In some implementations, the blood collection line 102 (for example, a portion of the blood collection line 102 positioned within channel 252 of peristaltic pump 250) comprises an internal diameter that is between approximately ⅛ inch and approximately 2 inch, for example, between approximately ¼ inch and approximately 1¾ inch, between approximately ½ inch and approximately 1½ inch, between approximately ¾ inch and approximately 1¼ inch, or between approximately ⅛ inch and approximately ½ inch, or any value or range within or bounded by any of these ranges or values, although values outside these values or ranges can be used in some cases. As another example, in some implementations, the blood collection line 102 (for example, a portion of the blood collection line 102 positioned within channel 252 of peristaltic pump 250) comprises an internal diameter that is approximately ¼ inch.

In some implementations, the plasma delivery line 108 (for example, a portion of the plasma delivery line 108 positioned within channel 252 of peristaltic pump 250) comprises an internal diameter that is between approximately 1/16 inch and approximately 1 inch, for example, between approximately ⅛ inch and approximately ¾ inch, between approximately ⅛ inch and approximately ½ inch, or between approximately 1/16 inch and approximately ¼ inch, or any value or range within or bounded by any of these ranges or values, although values outside these values or ranges can be used in some cases. As another example, in some implementations, the plasma delivery line 108 (for example, a portion of the plasma delivery line 108 positioned within channel 252 of peristaltic pump 250) comprises an internal diameter that is approximately ⅛ inch.

In some implementations, a ratio between an internal diameter of the blood collection line 102 (for example, a portion of the blood collection line 102 positioned within channel 252 of peristaltic pump 250) and an internal diameter of the plasma delivery line 108 (for example, a portion of the plasma delivery line 108 positioned within channel 252 of peristaltic pump 250) is between approximately 1 and approximately 5, for example, between approximately 1.5 and approximately 4.5, between approximately 2 and approximately 4, between approximately 2.5 and approximately 3.5, between approximately 1.5 and approximately 2.5, between approximately 1 and approximately 3, between approximately 1.1 and approximately 2.9, between approximately 1.2 and approximately 2.8, between approximately 1.3 and approximately 2.7, between approximately 1.4 and approximately 2.6, between approximately 1.5 and approximately 2.5, between approximately 1.6 and approximately 2.4, between approximately 1.7 and approximately 2.3, between approximately 1.8 and approximately 2.2, or between approximately 1.9 and approximately 2.1, or any value or range within or bounded by any of these ranges or values, although values outside these values or ranges can be used in some cases. As another example, a ratio between an internal diameter of the blood collection line 102 (for example, a portion of the blood collection line 102 positioned within channel 252 of peristaltic pump 250) and an internal diameter of the plasma delivery line 108 (for example, a portion of the plasma delivery line 108 positioned within channel 252 of peristaltic pump 250) is approximately 2.

In some implementations, a flow rate through the blood collection line 102 (for example, a portion of the blood collection line 102 positioned within channel 252 of peristaltic pump 250) to the blood separation device 106 is between approximately 10 mL/minute and approximately 550 mL/minute, for example, between approximately 50 mL/minute and approximately 500 mL/minute, between approximately 100 mL/minute and approximately 450 mL/minute, between approximately 150 mL/minute and approximately 400 mL/minute, between approximately 200 mL/minute and approximately 350 mL/minute, or between approximately 250 mL/minute and approximately 300 mL/minute, or any value or range within or bounded by any of these ranges or values, although values outside these values or ranges can be used in some cases. As another example, the flow rate through the blood collection line 102 (for example, a portion of the blood collection line 102 positioned within channel 252 of peristaltic pump 250) to the blood separation device 106 is approximately 280 mL/minute.

In some implementations, a flow rate through the blood collection line 102 (for example, a portion of the blood collection line 102 positioned within channel 252 of peristaltic pump 250) to the blood separation device 106 is at least approximately 5 mL/minute, at least approximately 10 mL/minute, at least approximately 20 mL/minute, at least approximately 50 mL/minute, at least approximately 100 mL/minute, at least approximately 150 mL/minute, at least approximately 200 mL/minute, or at least approximately 250 mL/minute, or any value or range within or bounded by any of these ranges or values, although values outside these values or ranges can be used in some cases. As another example, the flow rate through the blood collection line 102 (for example, a portion of the blood collection line 102 positioned within channel 252 of peristaltic pump 250) to the blood separation device 106 is at least approximately 280 mL/minute. Additionally or alternatively, in some implementations, the flow rate through the blood collection line 102 (for example, a portion of the blood collection line 102 positioned within channel 252 of peristaltic pump 250) to the blood separation device 106 is no greater than approximately 500 mL/minute, no greater than approximately 450 mL/minute, no greater than approximately 400 mL/minute, no greater than approximately 350 mL/minute, or no greater than approximately 300 mL/minute.

In some implementations, a flow rate through the plasma delivery line 108 (for example, a portion of the plasma delivery line 108 positioned within channel 252 of peristaltic pump 250) to the plasma processing device 112 is between approximately 0 mL/minute and approximately 200 mL/minute, for example, between approximately 10 mL/minute and approximately 190 mL/minute, between approximately 20 mL/minute and approximately 180 mL/minute, between approximately 30 mL/minute and approximately 170 mL/minute, between approximately 40 mL/minute and approximately 160 mL/minute, between approximately 50 mL/minute and approximately 150 mL/minute, between approximately 60 mL/minute and approximately 140 mL/minute, between approximately 70 mL/minute and approximately 130 mL/minute, between approximately 80 mL/minute and approximately 120 mL/minute, between approximately 90 mL/minute and approximately 110 mL/minute, between approximately 50 mL/minute and approximately 100 mL/minute, or between approximately 80 mL/minute and approximately 90 mL/minute, or any value or range within or bounded by any of these ranges or values, although values outside these values or ranges can be used in some cases.

In some implementations, a flow rate through the plasma delivery line 108 (for example, a portion of the plasma delivery line 108 positioned within channel 252 of peristaltic pump 250) to the plasma processing device 112 is at least approximately 5 mL/minute, at least approximately 10 mL/minute, at least approximately 20 mL/minute, approximately 30 mL/minute, approximately 40 mL/minute, approximately 50 mL/minute, approximately 60 mL/minute, approximately 70 mL/minute, or approximately 80 mL/minute, or any value or range within or bounded by any of these ranges or values, although values outside these values or ranges can be used in some cases. Additionally or alternatively, in some implementations, the flow rate through the plasma delivery line 108 (for example, a portion of the plasma delivery line 108 positioned within channel 252 of peristaltic pump 250) to the plasma processing device 112 is no greater than approximately 150 mL/minute, no greater than approximately 140 mL/minute, no greater than approximately 130 mL/minute, no greater than approximately 120 mL/minute, no greater than approximately 110 mL/minute, no greater than approximately 100 mL/minute, or no greater than approximately 90 mL/minute.

In some implementations, a flow rate through the plasma delivery line 108 (to the plasma processing device 112) is between approximately 5% and approximately 50% of a flow rate through the blood collection line 102 (to the blood separation device 106). For example, the flow rate through the plasma delivery line 108 (to the plasma processing device 112) can be between approximately 10% and approximately 45%, between approximately 15% and approximately 40%, between approximately 20% and approximately 35%, between approximately 25% and approximately 30%, or between approximately 20% and approximately 30% the flow rate through the blood collection line 102 (to the blood separation device 106), or any value or range within or bounded by any of these ranges or values, although values outside these values or ranges can be used in some cases.

With reference to FIG. 1 and as mentioned previously, the blood processing system 100 can include a return line 110 connected to the blood separation device 106 (for example, to outlet port 106c). As discussed above, the blood separation device 106 can separate the received blood (which may be referred to as “whole blood” prior to separation) from blood collection line 102 into various portions to separate components of the blood, for example, a first fraction including a portion of the plasma (that can be directed to plasma delivery line 108) and a second fraction including a portion of the plasma and also one or more cellular components (for example, red blood cells, white blood cells, and/or platelets). Such second fraction can be directed to return line 110, for example, via outlet port 106c.

As also shown in FIG. 1, the blood processing system 100 can include a return line 114 that is connected to the plasma processing device 112 (for example, outlet port 112b). The return line 114 can receive the first fraction after at least a portion of the plasma in such first fraction is processed by the plasma processing device 112. For example, where the plasma processing device 112 is an adsorption device, the return line 114 can receive the first fraction after at least a portion of the plasma in such first fraction is adsorbed by the plasma processing device 112, for example, by one or more absorbent materials such as any of those discussed herein. In some implementations, the blood processing system a particle filter 120 in and/or along the return line 114 that can be utilized to remove (for example, trap) fine particles.

As shown in FIG. 1, the return lines 110, 114 can join one another so that the second fraction flowing through return line 110 and the first fraction flowing through return line 114 (after being processed by plasma processing device 112) can be combined and returned to the subject 101. Such joining is represented in FIG. 1 at point 116. Return lines 110, 114 can be joined (for example, at point 116) via a branched connector having three branches (which may be referred to as “ports”), two of which can be connected to return lines 110, 114 and a third of which can be connected to an additional return line 124. Return line 124 can be connected to a blood source access device that can provide access to vasculature of subject 101 to allow the combined blood flowing through return line 124 to be delivered to the subject 101. In some implementations, blood processing system 100 includes a single blood source access device (such as a dual lumen catheter) that can be connected to both of blood collection line 102 and return line 124 to allow withdrawal of blood and delivery of processed blood, for example, simultaneously. In some variants, blood processing system 100 includes two, separate blood source access devices, one of which is connected to blood collection line 102 and which withdraws blood from the subject 101, and the other of which is connected to return line 124 and receives the processed, combined blood and delivers the same to the subject 101. In some of such variants, the two, separate blood source access devices can be positioned away from one another, in the same or in different portions of the subject's body (for example, arm). In some variants, the return lines 110, 114 are not connected to one another (for example, via a branched connector) and are each connected to separate blood source access devices that deliver the first and second fractions of the blood to a subject separately from one another.

In some implementations, blood processing system 100 includes a bubble or leak detector 122. Bubble or leak detector 122 can be positioned along return line 124, or another location within blood processing system 100. In some implementations, blood processing system 100 includes one or more pressure sensors configured to detect one or more pressure values in system 100. For example, blood processing system 100 can include a pressure sensor 126a located along blood collection line 102 (for example, at or proximate to blood separation device 106 and/or inlet 106a) for detecting pressure of the blood flowing to the blood separation device 106. Additionally or alternatively, blood processing system 100 can include a pressure sensor 126b located along plasma delivery line 108 (for example, at or proximate to plasma processing device 112 and/or inlet 112a) for detecting pressure of the plasma flowing to the plasma processing device 112. Although not illustrated in FIG. 1, blood processing system 100 can additionally or alternatively include a pressure sensor along return line 110 (for example, at or proximate to blood separation device 106 and/or inlet 106c), a pressure sensor along return line 114 (for example, at or proximate to plasma processing device 112 and/or outlet 112b, and/or a pressure sensor along return line 124).

In some implementations, blood processing system 100: does not include a pump along return line 110; does not include a pump along return line 114; and/or does not include a pump along return line 124. In some implementations, blood processing system 100, does not include a pump downstream (given the flow direction illustrated in FIG. 1) of the plasma processing device 112. In some implementations, blood processing system 100, does not include a pump downstream (given the flow direction illustrated in FIG. 1) of the outlet 106c. In some implementations, the pump 104 is the only pump in an extracorporeal blood circuit formed by blood processing system 100 that separates at least a portion of plasma from a subject's blood, processes (for example, adsorbs) such separated plasma, and returns such processed plasma along with remainder blood components (for example, one or more cellular blood components) to the subject.

Blood processing system 100 can include a controller 130, schematically illustrated in FIG. 1. The controller 130 can be configured to control operation of pump 104. For example, the controller 130 can be utilized to control one or more operating characteristics of pump 104, such as speed, acceleration, time of operation, operational mode, among other characteristics. As discussed above, pump 104 can be a peristaltic pump, for example, similar or identical to peristaltic pump 250. In such implementations, the controller 130 can be configured to control rotational speed, acceleration, time of operation, operational mode, among other characteristics of a rotor of the pump 104 (which can be similar or identical to rotor 254 described above in some or many respects). The controller 130 can be part of pump 104 or can be separate from pump 104. In some implementations, the controller 130 and pump 104 are embodied in a device that can be operated by a user, for example, via a user interface and/or remotely operated by a user (for example, where the controller 130 is configured to wireless communication with a computing device operable by the user). The controller 130 can comprise a processor and/or a memory. In some implementations, the controller 130 is embodied in one or more printed circuit boards.

In some implementations where blood processing system 100 includes pressure sensor 126a and/or 126b (and/or any additional pressure sensors), the controller 130 is in communication with any or all of such pressure sensors and can control operation of pump 104 based upon one or more pressure values detected by any of such pressure sensors. For example, in some of such implementations, controller 130 can be configured to vary one or more operating characteristics (such as any of those mentioned above) based upon one or more pressure values detected by any of such pressure sensors. As another example, controller 130 can be configured to receive one or more pressure values from any of the above-described pressure sensors, compare such pressure values to one or more thresholds, and vary one or more operating characteristics (such as any of those mentioned above) based on such comparison. For example, controller 130 can be configured to receive one or more pressure values from pressure sensor 126a and/or pressure sensor 126b and change a speed of pump 104 (for example, reduce such speed) and/or turn the pump 104 off where such pressure values are greater than a threshold (for example, a maximum pressure threshold). In some implementations, the controller 130 can be configured to receive one or more pressure values from each of pressure sensor 126a and pressure sensor 126b, compare the received one or more pressure values from each of pressure sensors 126a, 126b with each other and/or with one or more thresholds, and change one or more operating characteristics of the pump 104 (for example, change a speed of the pump 104 or turn the pump 104 off) based on such comparison(s). Such implementations can advantageously allow blood processing system 100 to dynamically modify flow through the blood collection and plasma delivery lines 102, 108, blood separation device 106, plasma processing device 112, and/or any other components in system 100 to facilitate optimal conditions and/or to protect the integrity of any of such components. For example, such implementations can facilitate optimal performance of the blood separation device 106 and/or the plasma processing device 112.

Although FIG. 1 illustrates lines 102, 108, 110, 114 having one or more bends (for example, perpendicular bends), such schematic illustration of lines 102, 108, 110, 114 is not intended to be limiting nor is such schematic illustration of lines 102, 108, 110, 114 intended to convey that any lines 102, 108, 110, 114 are required to have bends in practical implementations during use. Further, such schematic illustration of lines 102, 108, 110, 114 is not intended to convey that any of lines 102, 108, 110, 114 must comprise more than one single tube. As discussed above, any of lines 102, 108, 110, 114 can comprise a single tube, or alternatively, a plurality of connected tubes.

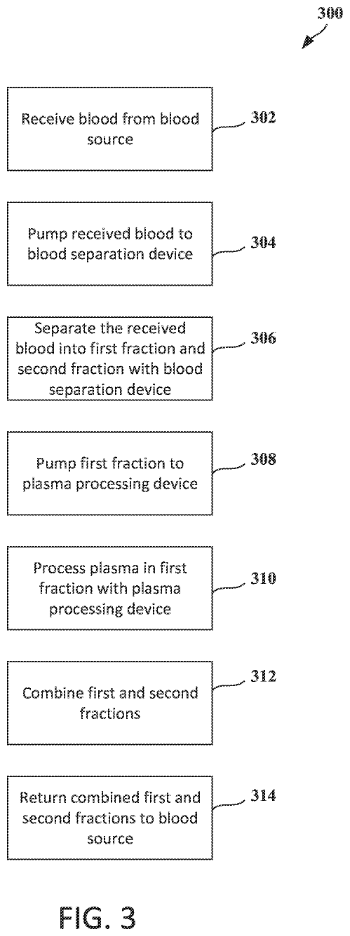

FIG. 3 illustrates a method 300 of processing blood, for example, to remove one or more components from blood plasma and return the processed blood to a subject. The method 300 can be utilized to treat various conditions of a subject, for example, in relation to atherosclerosis, cancer, degenerative and/or autoimmune diseases, among others. Method 300 can begin at step 302 where blood is received from a blood source. Such blood source can be vasculature of a subject (for example, a vein in the subject's arm). Alternatively, in some implementations, the blood source is a container that includes blood (for example, from the subject) that is stored extracorporeally outside the subject's body. The blood can be received from the blood source via a blood source access device such as any of those. Where the blood source is a container, the blood source access device can be any device capable of withdrawing blood from such container. The blood can be received and carried by a blood collection line of a blood processing system, such as blood collection line 102 described herein. The received blood can be pumped via a pump at step 304, for example, with a pump similar or identical to any of the pumps described herein (such as pump 104 and/or pump 250). Step 304 can involve causing the received blood to flow through a blood collection line (such as blood collection line 102) at a flow rate, for example, to a blood separation device such as blood separation device 106.

At step 306, the received blood (which may be referred to as “whole blood” prior to separation) can be separated by a blood separation device (such as blood separation device 106) into a plurality of fractions, such as a first fraction and a second fraction. As discussed elsewhere herein, such “first fraction” can include a portion of the plasma from the received blood and such “second fraction” can include a portion of the plasma from the received blood and one or more cellular components (for example, red blood cells, white blood cells, and/or platelets). Step 306 may involve separating the received blood into such first and second fractions such that the first fraction comprises plasma representing a first percentage of a total volume of the received blood entering the blood separation device and the separated, second fraction of the received blood can comprise a second percentage of such total volume. Such first and second percentages can be any of the values or ranges discussed previously.

The method 300 may further comprise directing such first fraction towards and/or out of a first outlet port of the blood separation device (for example, outlet port 106b of blood separation device 106) and directing such second fraction towards and/or out of a second outlet port of the blood separation device (for example, outlet port 106c of blood separation device 106). The method 300 may further comprise delivering the first fraction to a plasma delivery line (such as plasma delivery line 108), through such plasma delivery line, and to a plasma processing device (such as plasma processing device 112). As shown in FIG. 3, at step 308, the first fraction can be pumped to the plasma processing device, for example, via such plasma delivery line. Such pumping can be performed by the same pump mentioned above with respect to step 304. Further, step 304 and step 308 can be simultaneously performed such that the same (single) pump causes the received blood to flow through the blood collection line (for example, blood collection line 102) to blood separation device and, at the same time, causes the first fraction to flow through the plasma delivery line to the plasma processing device. The method 300 can further comprise arranging portions of such blood collection line and plasma delivery line inside a portion of such pump, for example, arranging such portions within the same channel in a peristaltic pump as described elsewhere herein. In some implementations, such portions of the blood collection line and plasma delivery line arranged within the same channel in a peristaltic pump comprise tubes having different internal diameters. Such implementations can therefore facilitate differential flow rates/volumes through the blood collection line and plasma delivery line, and in turn, differential flow rates/volumes to the blood separation device (which receives blood from the blood collection line) and the plasma processing device (which receives the first fraction of the blood from the plasma delivery line).

At step 310, the first fraction is processed (which may also be referred to as “purified”) by a plasma processing device (which may also be referred to as a “plasma purification device”). Such plasma processing device can be similar or identical to plasma processing device 112 discussed elsewhere herein. The plasma processing at step 310 can involve any of a variety of techniques for removing one or more substances (which may also be referred to as “components” herein) from the received plasma. For example, step 310 can involve filtering and/or adsorbing at least a portion of the plasma in the first fraction to remove one or more substances therefrom. In some implementations, step 310 comprises adsorbing at least a portion of the plasma in the first fraction using one or more adsorptive materials suitable for removing one or more components from the plasma with an adsorption device. Step 310 can comprise utilizing any of the methods, systems, and/or devices described in U.S. Pat. No. 11,123,465, titled “Methods of Using Thermally Expanded Graphite to Remove Proteins from Blood and to Treat Sepsis”, issued on Sep. 21, 2021, which is hereby incorporated by reference herein in its entirety.

At step 312, the first and second fractions can be combined. A return line (such as return line 110) can be connected to an outlet of the blood separation device (such as outlet 106c of blood separation device 106) and another return line (such as return line 114) can be connected to an outlet of the plasma processing device (such as outlet 112b of plasma processing device 112), and such return lines can be joined together to combine the first and second fractions.

At step 314, the combined first and second fractions can be return to the blood source. In some implementations, the method further comprises carrying the combined first and second fractions to another return line (such as return line 124) that can be connected to the above-mentioned return lines connected to the blood separation and plasma processing devices, prior to return to the blood source. As discussed above, such blood source can be vasculature of a subject or be a container that extracorporeally stores blood.

### Additional Considerations and Terminology

Although this invention has been disclosed in the context of certain preferred embodiments, it should be understood that certain advantages, features and aspects of the systems, devices, and methods may be realized in a variety of other embodiments. Additionally, it is contemplated that various aspects and features described herein can be practiced separately, combined together, or substituted for one another, and that a variety of combination and subcombinations of the features and aspects can be made and still fall within the scope of the invention. Furthermore, the systems and devices described above need not include all of the modules and functions described in the preferred embodiments.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

Although certain embodiments and examples have been described herein, it will be understood by those skilled in the art that many aspects of the systems and devices shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Any methods disclosed herein need not be performed in the order recited. The methods disclosed herein may include certain actions taken by a practitioner; however, they can also include any third-party instruction of those actions, either expressly or by implication.

What is claimed is:

1. A blood processing system comprising:
a blood collection line configured to carry blood;
a blood separation device connected to the blood collection line and configured to separate the blood into a first fraction and a second fraction, said first fraction comprising plasma and said second fraction comprising one or more cellular components of the blood;
a plasma delivery line connected to the blood separation device and configured to receive the first fraction of the blood;
a plasma processing device connected to the plasma delivery line, the plasma processing device configured to process the first fraction of the blood;
a pump, wherein a portion of the blood collection line and a portion of the plasma delivery line are positioned within the pump, and wherein the pump is configured to simultaneously cause the blood to flow through the blood collection line to the blood separation device and cause the first fraction of the blood to flow through the plasma delivery line to the plasma processing device;
a first return line connected to the plasma processing device and configured to receive the processed the first fraction of the blood; and
a second return line connected to the blood separation device and configured to receive the second fraction of the blood, wherein the second return line is connected to the first return line, thereby allowing the second fraction of the blood to be combined with the processed the first fraction of the blood.

2. The blood processing system of claim 1, wherein said pump is the only pump in the blood processing system.

3. The blood processing system of claim 1 or 2, wherein said pump is a peristaltic pump.

4. The blood processing system of claim 3, wherein said peristaltic pump comprises a channel and a rotor, wherein said portion of the blood collection line and said portion of the plasma delivery line are arranged within the channel, and wherein the rotor is configured to cause the received blood to flow through the blood collection line and cause the first portion of the blood to flow through the plasma delivery line.

5. The blood processing system of claim 4, wherein said rotor comprises one or more rollers configured to compress portions of the blood collection and plasma delivery lines during rotation of the rotor.

6. The blood processing system of claim 5, wherein each of the one or more rollers are configured to simultaneously compress said portions of the blood collection and plasma delivery lines during rotation of the rotor.

7. The blood processing system of any of claims 4-6, wherein said rotor comprises at least two rollers.

8. The blood processing system of any of claims 4-7, wherein each of said portion of the blood collection line and said portion of the plasma delivery line is arranged in a generally U-shape within said channel.

9. The blood processing system of any of claims 4-8, wherein at least a portion of said channel of said peristaltic pump comprises a generally U-shape.

10. The blood processing system of any of claims 4-9, wherein said channel is the only channel of said peristaltic pump.

11. The blood processing system of any of claims 4-10, wherein said portion of the blood collection line and said portion of the plasma delivery line are arranged to contact one another within the channel.

12. The blood processing system of any of claims 4-11, wherein:
said portion of the blood collection line arranged within said channel comprises a first tube having a first internal diameter;
said portion of the plasma delivery line arranged within said channel comprises a second tube having a second internal diameter;
said first and second internal diameters are different; and
said peristaltic pump is configured to simultaneously cause the blood flowing through the blood collection line to flow at a first flow rate and cause the first fraction of the blood flowing through the plasma delivery line to flow at a second flow rate that is different than the first flow rate.

13. The blood processing system of claim 12, wherein the second flow rate is less than the first flow rate.

14. The blood processing system of claim 12 or 13, wherein a ratio between the first internal diameter and the second internal diameter is between approximately 1 and approximately 3.

15. The blood processing system of claim 14, wherein the ratio between the first internal diameter and the second internal diameter is approximately 2.

16. The blood processing system of any of claims 12-15, wherein the second flow rate is between approximately 20% and approximately 30% of the first flow rate.

17. The blood processing system of any of claims 12-16, wherein the second flow rate is no greater than approximately 100 mL/minute.

18. The blood processing system of any of claims 12-17, wherein the first flow rate is at least approximately 50 mL/minute.

19. The blood processing system of any of claims 1-18, wherein the blood separation device comprises:
an inlet port connected to the blood collection line;
a first outlet port connected to the plasma delivery line; and
a second outlet port connected to the second return line.

20. The blood processing system of any of claims 1-19, further comprising a third return line connected to both of the first and second return lines, wherein the third return line is configured to deliver the second fraction of the blood to the subject along with the processed first fraction of the blood.

21. The blood processing system of claim 20, further comprising a branched connector connecting the first, second, and third return lines together.

22. The blood processing system of claim 20 or 21, further comprising a blood source access device configured to connect to the third return line and deliver the second fraction of the blood to the subject along with the processed first fraction of the blood.

23. The blood processing system of claim 22, wherein said blood source access device comprises dual lumen catheter, wherein the blood collection line is configured to connect to a first port of the dual lumen catheter and wherein the third return line is configured to connect to a second port of the dual lumen catheter.

24. The blood processing system of any of claims 1-23, further comprising a controller in communication with the pump and configured to change one or more operating characteristics of the pump.

25. The blood processing system of claim 24, wherein said one or more operating characteristics comprises a speed of the pump.

26. The blood processing system of claim 25, wherein the pump is a peristaltic pump comprising a rotor, and wherein said one or more operating characteristics comprises a rotational speed of the rotor of the peristaltic pump.

27. The blood processing system of any of claims 24-26, further comprising a first pressure sensor configured to detect one or more pressure values of the first fraction prior to entering the plasma processing device, wherein the controller is configured to receive said one or more pressure values and change said one or more operating characteristics of the pump based upon said one or more pressure values.

28. The blood processing system of claim 27, wherein the controller is further configured to compare said one or more pressure values to one or more thresholds and change said one or more operating characteristics of the pump based upon said comparison.

29. The blood processing system of claim 27 or 28, further comprising a second pressure sensor configured to detect one or more pressure values of the blood prior to entering the blood separation device, wherein the controller is configured to receive said one or more pressure values from the second pressure sensor and change said one or more operating characteristics of the pump based upon said one or more pressure values received from said second pressure and based upon said one or more pressure values received from said first pressure sensor.

30. The blood processing system of claim 29, wherein the controller is further configured to compare said one or more pressure values received from the second pressure sensor to one or more thresholds and change said one or more operating characteristics of the pump based upon said comparison.

31. The blood processing system of claim 29 or 30, wherein the controller is further configured to compare said one or more pressure values received from said first pressure sensor with said one or more pressure values received from said second pressure sensor and change said one or more operating characteristics of the pump based upon said comparison.

32. The blood processing system of any of claims 1-31, wherein the blood separation device comprises a centrifugal separator.

33. The blood processing system of any of claims 1-32, wherein the blood separation device comprises one or more membranes.

34. The blood processing system of claim 33, wherein each of the one or more membranes comprises a pore size between approximately 0.2 μm and approximately 1 μm.

35. The blood processing system of any of claims 1-34, wherein said first fraction comprises a first percentage of a total volume of the blood entering the blood separation device and said second fraction comprises a second percentage of said total volume.

36. The blood processing system of claim 35, wherein said first percentage is less than said second percentage.

37. The blood processing system of claim 35 or 36, wherein said first percentage is between approximately 5% and approximately 50% of said total volume.

38. The blood processing system of any of claims 35-37, wherein said first percentage is between approximately 20% and approximately 30% of said total volume.

39. The blood processing system of any of claims 1-38, wherein said second fraction of the blood comprises plasma and said one or more cellular components.

40. The blood processing system of any of claims 1-39, wherein substantially an entirety of said first fraction comprises plasma.

41. The blood processing system of any of claims 1-40, wherein said one or more cellular components comprises at least one of white blood cells, red blood cells, and platelets.

42. The blood processing system of any of claims 1-41, wherein said blood collection line comprises one or more tubes.

43. The blood processing system of any of claims 1-41, wherein said blood collection line comprises a single tube.

44. The blood processing system of any of claims 1-41, wherein said plasma delivery line comprises one or more tubes.

45. The blood processing system of any of claims 1-41, wherein said plasma delivery line comprises a single tube.

46. The blood processing system of any of claims 1-45, wherein said plasma processing device is an adsorption device configured to adsorb at least a portion of the plasma of the first fraction of the blood to remove one or more substances from the first fraction of the blood.

47. The blood processing system of claim 46, wherein said adsorption device is in the form of a cartridge or column.

48. The blood processing system of claim 46 or 47, wherein the adsorption device comprises one or more absorptive materials selected from the group consisting of thermally expanded graphite (TEG), graphene nanoplatelets (GNPs), polymer derived ceramic carbide-derived carbon (PDC-CDC), an ion exchange resin, and a non-ion exchange resin.

49. The blood processing system of any of claims 1-48, wherein said blood collection line is configured to receive blood from a blood source.

50. The blood processing system of claim 49, wherein said blood source is a container.

51. The blood processing system of claim 49, further comprising a blood source access device connected to the blood collection line, wherein said blood source comprises one of a vein, an artery, or an arteriovenous fistula of a subject, and wherein said blood source access device is configured to withdraw said blood from the subject's vein, artery, or arteriovenous fistula.

52. The blood processing system of any of claims 1-51, wherein said pump is the only pump arranged along the blood collection line and the plasma delivery line.

53. The blood processing system of any of claims 1-51, wherein said pump is the only pump arranged between the blood separation device and the plasma processing device.

54. A blood processing system comprising:
a blood collection line configured to carry blood;
a blood separation device comprising an inlet port connected to the blood collection line, a first outlet port, and a second outlet port, wherein the blood separation device is configured to:
receive the blood from the blood collection line via the inlet port;
separate the blood into a first fraction and a second fraction, said first fraction comprising plasma and said second fraction comprising one or more cellular components of the blood;
direct the first fraction of the blood to the first outlet port; and
direct the second fraction of the blood to the second outlet port;
a plasma delivery line connected to the first outlet port of the blood separation device and configured to receive the first fraction of the blood;
an adsorption device comprising an inlet port connected to the plasma delivery line and an outlet port, wherein the adsorption device is configured to adsorb at least a portion of the plasma of the first fraction of the blood to remove one or more substances from the first fraction of the blood;
a peristaltic pump comprising a channel and a rotor, wherein:
a portion of the blood collection line comprising a first internal diameter and a portion of the plasma delivery line comprising a second internal diameter are arranged adjacent one another within the channel;
the first and second internal diameters are different from one another; and
the rotor is configured to cause the blood to flow through the blood collection line to the blood separation device at a first flow rate and cause the first portion of the blood to flow through the plasma delivery line to the adsorption device at a second flow rate that is less than the first flow rate;
a first return line connected to the outlet of the adsorption device and configured to receive the processed the first fraction of the blood; and
a second return line connected to second outlet port of the blood separation device and configured to receive the second fraction of the blood, wherein the second return line is connected to the first return line, thereby allowing the second fraction of the blood to be combined with the processed the first fraction of the blood.

55. The blood processing system of claim 54, wherein the rotor is configured to simultaneously cause the blood to flow through the blood collection line to the blood separation device at the first flow rate and the first portion of the blood to flow through the plasma delivery line to the adsorption device at the second flow rate.

56. The blood processing system of claim 54 or 55, said peristaltic pump is the only pump in the blood processing system.

57. The blood processing system of any of claims 54-55, wherein said peristaltic pump is the only pump arranged along the blood collection line and the plasma delivery line.

58. The blood processing system of any of claims 54-55, wherein said peristaltic pump is the only pump arranged between the blood separation device and the plasma processing device.

59. The blood processing system of any of claims 54-58, wherein said rotor is configured to rotate and comprises one or more rollers configured to compress said portions of the blood collection and plasma delivery lines during rotation of the rotor.

60. The blood processing system of claim 59, wherein each of the one or more rollers are configured to simultaneously compress said portions of the blood collection and plasma delivery lines during rotation of the rotor.

61. The blood processing system of claim 59 or 60, wherein said rotor comprises at least two rollers.

62. The blood processing system of any of claims 54-61, wherein each of said portion of the blood collection line and said portion of the plasma delivery line is arranged in a generally U-shape within said channel.

63. The blood processing system of any of claims 54-62, wherein at least a portion of said channel of said peristaltic pump comprises a generally U-shape.

64. The blood processing system of any of claims 54-63, wherein said channel is the only channel of said peristaltic pump.

65. The blood processing system of any of claims 52-64, wherein said portion of the blood collection line and said portion of the plasma delivery line are arranged to contact one another within the channel.

66. The blood processing system of any of claims 54-65, wherein said portion of the blood collection line and said portion of the plasma delivery line each comprise a tube.

67. The blood processing system of any of claims 54-66, wherein a ratio between the first internal diameter and the second internal diameter is between approximately 1 and approximately 3.

68. The blood processing system of claim 67, wherein the ratio between the first internal diameter and the second internal diameter is approximately 2.

69. The blood processing system of any of claims 54-68, wherein the second flow rate is between approximately 20% and approximately 30% of the first flow rate.

70. The blood processing system of any of claims 54-69, wherein the second flow rate is no greater than approximately 100 mL/minute.

71. The blood processing system of any of claims 54-60, wherein the first flow rate is at least 50 mL/minute.

72. The blood processing system of any of claims 54-71, further comprising a controller in communication with the peristaltic pump and configured to change one or more operating characteristics of the peristaltic pump.

73. The blood processing system of any of claims 54-72, wherein the blood separation device comprises one or more membranes.

74. The blood processing system of claim 73, wherein each of the one or more membranes comprises a pore size between approximately 0.2 μm and approximately 1 μm.

75. The blood processing system of any of claims 54-74, wherein said first fraction comprises a first percentage of a total volume of the blood entering the blood separation device and said second fraction comprises a second percentage of said total volume, and wherein said first percentage is less than said second percentage.

76. The blood processing system of claim 75, wherein said first percentage is between approximately 20% and approximately 30% of said total volume.

77. The blood processing system of any of claims 54-76, wherein each of said blood collection line and said plasma delivery line comprises a single tube.

78. The blood processing system of any of claims 54-77, wherein said adsorption device is in the form of a cartridge or column.

79. The blood processing system of any of claims 54-78, wherein the adsorption device comprises one or more absorptive materials selected from the group consisting of thermally expanded graphite (TEG), graphene nanoplatelets (GNPs), polymer derived ceramic carbide-derived carbon (PDC-CDC), an ion exchange resin, and a non-ion exchange resin.

80. A blood processing system comprising:
a first blood transport line configured to carry blood to a blood separation device, the blood separation device configured to separate the blood into a first fraction and a second fraction, said first fraction comprising plasma and said second fraction comprising one or more cellular components of the blood;
a second blood transport line connected to the blood separation device and configured to deliver the first fraction of the blood to an adsorption device, the adsorption device configured to adsorb at least a portion of the plasma of the first fraction of the blood to remove one or more substances from the first fraction of the blood; and
a peristaltic pump configured to simultaneously cause the blood to flow through the first blood transport line to the blood separation device and the first fraction of the blood to flow through the second blood transport line to the adsorption device.

81. The blood processing system of claim 80, wherein said peristaltic pump is the only pump in the blood processing system.

82. The blood processing system of claim 80, wherein no other pumps cause the blood to flow through the first blood transport line to the blood separation device and the first fraction of the blood to flow through the second blood transport line to the adsorption device.

83. The blood processing system of any of claims 80-82, wherein a portion of the first blood transport line and a portion of the second blood transport line are positioned within the peristaltic pump.

84. The blood processing system of claim 83, wherein the peristaltic pump comprises a channel and said portions of the first and second blood transport lines are positioned within said channel.

85. The blood processing system of claim 84, wherein said peristaltic pump further comprises a rotor, said rotor configured to rotate and comprising one or more rollers, said one or more rollers configured to compress said portions of the first and second blood transport lines during rotation of the rotor.

86. The blood processing system of claim 85, wherein each of the one or more rollers are configured to simultaneously compress said portions of the first and second blood transport lines during rotation of the rotor.

87. The blood processing system of claim 85 or 86, wherein said rotor comprises at least two rollers.

88. The blood processing system of any of claims 85-87, wherein each of said portions of the first and second blood transport lines is arranged in a generally U-shape within said channel.

89. The blood processing system of any of claims 84-88, wherein said channel is the only channel in said peristaltic pump.

90. The blood processing system of any of claims 84-89, wherein said channel comprises a generally U-shape.

91. The blood processing system of any of claims 83-90, wherein each of said portions of the first and second blood transport lines comprises a tube.

92. The blood processing system of any of claims 83-91, wherein said portion of the first blood transport line comprises a tube having a first internal diameter and said portion of the second transport line comprises a tube having a second internal diameter that is less than the first internal diameter.

93. The blood processing system of claim 92, wherein a ratio between the first internal diameter and the second internal diameter is between approximately 1 and approximately 3.

94. The blood processing system of claim 92, wherein a ratio between the first internal diameter and the second internal diameter is approximately 2.

95. The blood processing system of any of claims 80-94, wherein said peristaltic pump is configured to simultaneously cause the blood to flow through the first blood transport line to the blood separation device at a first flow rate and the first fraction of the blood to flow through the second blood transport line to the adsorption device at a second flow rate that is less than the first flow rate.

96. The blood processing system of claim 95, wherein the second flow rate is between approximately 20% and approximately 30% of the first flow rate.

97. A method of processing blood comprising:
carrying blood with a blood collection line;
separating the blood into a first fraction and a second fraction with a blood separation device, said first fraction comprising plasma and said second fraction comprising one or more cellular components of the blood;
delivering the first fraction of the blood to a plasma processing device with a plasma delivery line;
simultaneously pumping, with a single pump, the blood through the blood collection line to the blood separation device and the first fraction of the blood to the plasma processing device, wherein a portion of the blood collection line and a portion of the plasma delivery line are positioned within the pump;
processing the first fraction of the blood with the plasma processing device; and
combining said second fraction of the blood with the processed first fraction of the blood.

98. The method of claim 97, further comprising receiving, from a blood source access device, the blood with the blood collection line.

99. The method of claim 98, wherein said receiving, from the blood source access device, the blood with the blood collection line comprises receiving the blood when the blood source access device is secured to a subject.

100. The method of any of claims 97-99, further comprising delivering said second fraction of the blood with the processed first fraction of the blood to a subject.

101. The method of any of claims 97-99, further comprising:
carrying the processed the first fraction of the blood through a first return line after the processed first fraction of the blood exits the plasma processing device;
carrying the second fraction of the blood through a second return line after the second fraction exits the blood separation device;
wherein said combining said second fraction of the blood with the processed first fraction of the blood comprises:
delivering the processed first fraction of the blood through the first return line to a third return line; and
delivering the second fraction of the blood through the second return line to the third return line; and
wherein the third return line is connected to the first and second return lines.

102. The method of claim 101, further comprising delivering the combined second fraction of the blood and the processed first fraction of the blood to a subject with a blood source access device.

103. The method of claim 101 or 102, wherein said combining said second fraction of the blood with the processed first fraction of the blood further comprises allowing said second fraction of the blood and the processed first fraction of the blood to flow through a branched connector connected to the first, second, and third return lines.

104. The method of any of claims 97-103, wherein said simultaneously pumping with the single pump further comprises simultaneously causing:
the blood flowing through the blood collection line to flow at a first flow rate; and
the first fraction of the blood flowing through the plasma delivery line to flow at a second flow rate that is different than the first flow rate.

105. The method of claim 104, wherein the second flow rate is less than the first flow rate.

106. The method of claim 104 or 105, wherein the second flow rate is between approximately 20% and approximately 30% of the first flow rate.

107. The method of any of claims 104-106, wherein the second flow rate is no greater than 100 mL/minute.

108. The method of any of claims 104-107, wherein the first flow rate is at least 50 mL/minute.

109. The method of any of claims 97-108, wherein:
said pump is a peristaltic pump comprising a channel and a rotor;
the method further comprises positioning the portion of the blood collection line and the portion of the plasma delivery line within the channel; and
said simultaneously pumping comprises rotating the rotor to cause the blood to flow through the blood collection line and cause the first fraction of the blood to flow through the plasma delivery line to the plasma purification device.

110. The method of claim 109, wherein the rotor comprises one or more rollers, and wherein said simultaneously pumping further comprises causing each of the one or more rollers to simultaneously compress portions of the blood collection and plasma delivery lines during rotation of the rotor.

111. The method of claim 109 or 110, wherein said positioning said portion of the blood collection line and said portion of the plasma delivery line within the channel comprises arranging each of said portion of the blood collection line and said portion of the plasma delivery line in a generally U-shaped configuration within the channel.

112. The method of any of claims 109-111, wherein said positioning said portion of the blood collection line and said portion of the plasma delivery line within the channel comprises bending said portion of the blood collection line and said portion of the plasma delivery line and inserting bent portions of the blood collection and plasma delivery lines within the channel.

113. The method of any of claims 109-112, wherein said channel is the only channel of said peristaltic pump.

114. The method of any of claims 109-113, wherein said channel comprises a generally U-shape.

115. The method of any of claims 97-114, wherein said portion of the blood collection line comprises a tube having a first internal diameter, and wherein said portion of the plasma delivery line comprises a tube having a second internal diameter that is different than the first internal diameter.

116. The method of claim 115, wherein the second internal diameter is less than the first internal diameter.

117. The method of claim 115 or 116, wherein a ratio between the first internal diameter and the second internal diameter is between approximately 1 and approximately 3.

118. The method of any of claims 97-117, further comprising changing one or more operating characteristics of the pump with a controller.

119. The method of claim 118, wherein said changing said one or more operating characteristics of the pump comprises changing a speed of the pump.

120. The method of any of claims 97-119, wherein said blood separation device comprises one or more membranes, and wherein said separating said the blood into said first and second fractions comprises allowing the blood to flow through said one or more membranes.

121. The method of any of claims 97-120, wherein said first fraction comprises a first percentage of a total volume of the blood entering the blood separation device and said second fraction comprises a second percentage of said total volume.

122. The method of claim 121, wherein said first percentage is less than said second percentage.

123. The method of claim 121 or 122, wherein said first percentage is between approximately 5% and approximately 50% of said total volume.

124. The method of any of claims 121-123, wherein said first percentage is between approximately 20% and approximately 30% of said total volume.

125. The method of any of claims 97-124, wherein said second fraction of the blood comprises plasma and said one or more cellular components.

126. The method of any of claims 97-125, wherein substantially an entirety of said first fraction comprises plasma.

127. The method of any of claims 97-126, wherein said one or more cellular components comprises at least one of white blood cells, red blood cells, and platelets.

128. The method of any of claims 97-127, wherein said blood collection line comprises a single tube.

129. The method of any of claims 97-128, wherein said plasma delivery line comprises a single tube.

130. The method of any of claims 97-129, wherein said plasma processing device is an adsorption device configured to adsorb at least a portion of the plasma of the first fraction of the blood to remove one or more substances from the first fraction of the blood.

131. The method of any of claims 97-130, wherein said adsorption device is in the form of a cartridge or column.

132. The method of any of claims 97-131, wherein the adsorption device comprises one or more absorptive materials selected from the group consisting of thermally expanded graphite (TEG), graphene nanoplatelets (GNPs), polymer derived ceramic carbide-derived carbon (PDC-CDC), an ion exchange resin, and a non-ion exchange resin.
